(19) United States
(12) Patent Application Publication
(10) Pub. No.: US 2026/0098358 A1
(43) Pub. Date: Apr. 9, 2026

(54) TUMOR CELL PROFILING-BASED THERAPEUTIC TARGETING OF CANCER

(71) Applicant: Astrin Biosciences, Inc., St. Paul, MN (US)

(72) Inventors: Catalina Galeano-Garces, Forest Lake, MN (US); Justin Drake, Plymouth, MN (US); Kaylee Judith Kamalanathan, Woodbury, MN (US); Jiarong Hong, Eden Prairie, MN (US); Nicholas Heller, Minneapolis, MN (US); Badrinath R. Konety, Edina, MN (US); Jayant Parthasarathy, Excelsior, MN (US)

(73) Assignee: Astrin Biosciences, Inc., St. Paul, MN (US)

(21) Appl. No.: 19/113,971

(22) PCT Filed: Sep. 22, 2023
(86) PCT No.: PCT/US2023/074847

§ 371 Date: Mar. 21, 2025

Related U.S. Application Data
(60) Provisional application No. 63/408,975, filed on Sep. 22, 2022.

(51) Int. Cl. C40B 30/06; C12Q 1/6886; C40B 50/06; G01N 33/50

(52) U.S. Cl. CPC C40B 30/06; C12Q 1/6886; C40B 50/06; G01N 33/5011; G01N 33/5082; G01N 2570/00

(57) ABSTRACT

Metastasis is a major cause of fatality and expense in cancer. Many metastatic patients express calcified, fibrotic, or deep-seated (lymph/Jung/brain) lesions that are not easily profiled. For such patients, decision-making regarding treatment selection is population-based and heavily reliant on standards of care and are not tailored to the individual's tumor. The present disclosure provides methods of identifying treatment protocols for cancer patients using released tumor cells (RTCs). Method described herein include isolating/enriching RTCs, profiling RTCs and comparing RTCs to reference RTC profile libraries.

### PRIORITY

This application claims the benefit of U.S. Ser. No. 63/408,975, filed on Sep. 22, 2022, which is incorporated herein by reference in its entirety.

### BACKGROUND

Metastasis is a major cause of fatality and expense in cancer. Many metastatic patients express calcified, fibrotic, or deep-seated (lymph/lung/brain) lesions that are not easily profiled. For such patients, decision-making regarding treatment selection is population-based and heavily reliant on standards of care and are not tailored to the individual's tumor. There remains a need in the art to develop treatment protocols that are customized to the profile of the cancer in a patient.

### SUMMARY

The present disclosure provides methods of preparing a reference released tumor cell (RTC) profile library. The methods can comprise:
(i) isolating or enriching one or more released tumor cells (RTCs) from one or more biological samples one or more subjects to form isolated or enriched RTCs;
(ii) expanding each of the one or more isolated or enriched RTCs in three-dimensional (3D) cell culture to form expanded RTCs;
(iii) contacting or treating the expanded RTCs with one or more therapeutic agents or therapeutic modalities to identify expanded RTCs that are responsive to the one or more therapeutic agents or therapeutic modalities and expanded RTCs that are non-responsive to the one or more therapeutic agents or therapeutic modalities; and
(iv) conducting molecular profiling, morphologic profiling, or both molecular profiling and morphologic profiling of the expanded RTCs that are responsive and the expanded RTCs that are non-responsive to the at least one therapeutic agent or therapeutic modality, thereby generating a reference RTC profile library.

The methods described herein can be conducted completely ex vivo. The enriched RTCs can be enriched by one or more of a physical enrichment method and a biological enrichment method prior to step (ii). Step (iv) can further comprise conducting molecular profiling of the isolated RTCs and the RTCs expanded in 3D cell culture. The at least one therapeutic agent can be a small molecule, a biological agent, or an immunotherapeutic agent. The methods can further comprise incorporating at least one immune cell type isolated from the biological sample, into the 3D cell culture. The expanded RTCs can be expanded in 3D cell culture as organoids, spheroids, microtumors, or ex vivo tumors. The isolated or enriched RTCs can be circulating tumor cells (CTCs) or disseminated tumor cells (DTCs). The morphological profiling can comprise holographic profiling. The molecular profiling can be genomic profiling (e.g., single cell DNA sequencing, bulk DNA sequencing), transcriptomic profiling (e.g., single cell RNA sequencing, bulk RNA sequencing), or proteomic profiling (e.g., single cell proteomics profiling, bulk proteomics profiling). The biological sample can be a blood sample, a serum sample, a plasma sample, a saliva sample, a fecal sample, a urine sample, a cerebrospinal fluid sample, an interstitial fluid sample, a peritoneal fluid sample, a pleural fluid sample, a cyst fluid sample, a semen sample, a mucous sample, a sputum sample, an intestinal fluid sample, a lymphatic fluid sample, a fine-needle aspirated tissue sample, or a bone marrow sample.

An aspect provides methods of determining a treatment protocol for a cancer patient. The methods can comprise:
(i) isolating RTCs from a biological sample of the cancer patient to form isolated RTCs;
(ii) conducting molecular profiling, morphologic profiling, or both molecular profiling and morphologic profiling of the isolated RTCs from the cancer patient to establish a molecular profile, a morphologic profile, or both molecular profile and morphologic profile of the isolated RTCs; and
(iii) comparing the molecular profile, morphologic profile, or both molecular profile and morphologic profile of the isolated RTCs from the cancer patient to a reference RTC profile library as described herein to identify a treatment protocol.

The isolated RTCs from the biological sample of the cancer patient can be enriched by one or more of a physical enrichment method or a biological enrichment method prior to step (ii). The RTCs from the cancer patient can be expanded as RTCs in 3D cell culture to form isolated, expanded RTCs prior to step (ii). The isolated, expanded RTCs can be expanded in 3D cell culture as organoids, spheroids, microtumors, or ex vivo tumors prior to step (ii). The isolated RTC can be a circulating tumor cell (CTC) or a disseminated tumor cell (DTC). The methods can further comprise incorporating at least one immune cell type isolated from the biological sample, into the 3D cell culture. The molecular profiling can be genomic profiling (e.g., single cell DNA sequencing, bulk DNA sequencing), transcriptomic profiling (e.g., single cell RNA sequencing, bulk RNA sequencing), or proteomic profiling (e.g., single cell proteomics profiling, bulk proteomics profiling). The morphological profiling can comprise holographic profiling. The biological sample can be a blood sample, a serum sample, a plasma sample, a saliva sample, a fecal sample, a urine sample, a cerebrospinal fluid sample, an interstitial fluid sample, a peritoneal fluid sample, a pleural fluid sample, a cyst fluid sample, a semen sample, a mucous sample, a sputum sample, an intestinal fluid sample, a lymphatic fluid sample, a fine-needle aspirated tissue sample or a bone marrow sample. The methods can further comprise comparing the molecular profile of the isolated RTCs from the cancer patient to a molecular profile of a reference cancer cell or a population thereof. The reference cancer cell can be a cancer cell line cell, a cancer biopsy cell, or a patient-derived xenograft cell.

### BRIEF DESCRIPTION OF THE DRAWINGS

Various objectives, features, and advantages of the disclosed subject matter can be more fully appreciated with reference to the following detailed description of the disclosed subject matter when considered in connection with the following drawings, in which like reference numerals identify like elements.

FIG. 1 provides a method of preparing a reference RTC profile library and identifying a treatment protocol according to the present disclosure.

### DETAILED DESCRIPTION

The details of one or more variations of the subject matter described herein are set forth in the accompanying drawings and the description below. Other features and advantages of the subject matter described herein will be apparent from the description and drawings, and from the claims. The disclosed subject matter is not, however, limited to any particular disclosed aspect.

### Overview

Over half of the patients diagnosed with cancer have mutations that cannot be directly addressed by a matched targeted therapy. The problem is particularly acute in patients who have failed a first round of anti-cancer treatment resulting in a recurrence of the cancer. Continuous clonal evolution leading to tumor heterogeneity contributes to treatment resistance and relapse of the cancer due to dissemination of the disease.

Released tumor cells (RTCs) are extremely rare cells shed from tumors. Analysis of RTCs can be used as a surrogate or to complement invasive tissue biopsies, which can be uncomfortable, has side-effects and is not always successful.

The present disclosure provides methods for utilizing the activity signatures of metastasis causing released tumor cells (RTCs) to reveal a real-time window into the tumor's heterogeneity and activated pathways. Furthermore, the surface protein expression of these cells could be used directly identify the most promising cellular and immunotherapies for that individual.

### Released Tumor Cells (RTCs)

RTCs can be defined as cancer cells that have been shed by or separated from a tumor and that migrate into surrounding tissue and/or a body fluid. RTCs can be an important biomarker for disease and represent a powerful tool to study tumor progression and evolution. RTCs can be released from tumors by active or passive mechanisms. RTCs isolated from a subject can be present as single cells or as clusters of 2 or more cells. In some aspects, RTCs can have to potential to seed distant organs and form additional tumors.

In some aspects, RTCs can be circulating tumor cells (CTCs). As used herein, CTCs refer to tumor cells that have been shed into the vasculature or lymphatic system.

In some aspects, the RTCs can be disseminated tumor cells (DTCs). As used herein, DTCs refer to tumor cells that have been shed from a tumor, which have subsequently landed at a second site in the same subject. DTCs can form a tumor at the second site in the subject.

### Methods of the Disclosure

The present disclosure provides method for therapeutic targeting of cancer based on profiling released tumor cells. As used herein, “profiling”, refers to the measurement of at least one feature or property of a cell such as, but not limited to, a molecular feature, or a morphological feature. Molecular features can include analysis of the DNA, RNA, protein, enzymatic activity, and/or metabolites of the cells. Morphological features can include cell shape, size, structure, form, or any other physical attributes of the cell.

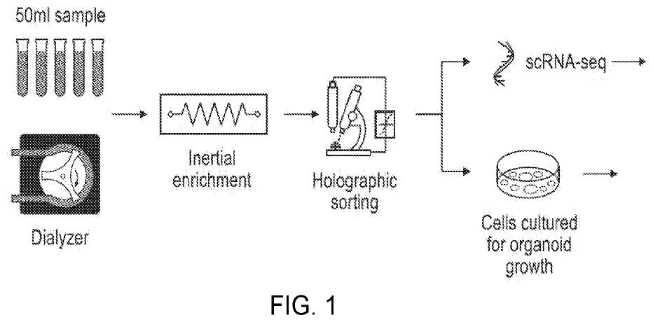

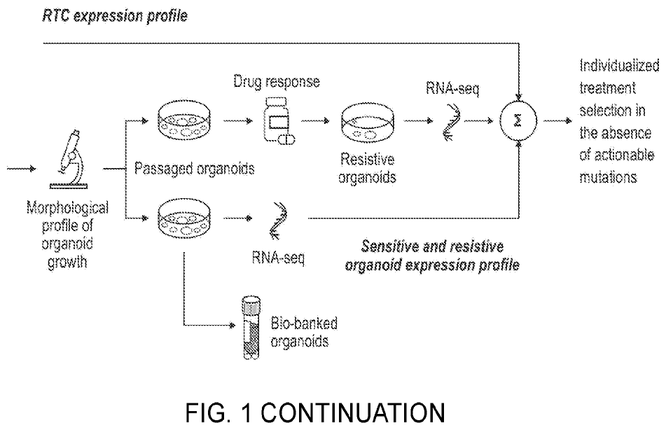

In some aspects, the methods can involve preparing a reference RTC profile library. The reference RTC profile library preparation can involve isolating and enriching RTCs from a biological sample. See, e.g., FIG. 1. For example, cells can be captured using a dialyzer. Cells can be subjected to inertial enrichment. Holographic imaging can be used to isolate RTCs. See WO2021155322; US Publ. No. 2023/004052, which are incorporated herein by reference.

A reference RTC profile library can be obtained from RTCs from a multitude of patients (e.g., 2, 5, 10, 20, 50, 100 or more) having, e.g., a specific type of cancer, RTCs from cancer patients all having a specific type of treatment, RTCs from cancer patients in general, RTCs from patients all having a related type of cancer (i.e., all types or kidney cancers or all types of breast cancers), RTCs from patients of a certain age (e.g., child, teen adult, or elderly patients), RTCs from patients having a particular stage of cancer, or RTCs from patients of one particular race. A reference RTC profile can also be obtained from one patient, for example, RTCs obtained from a patient before treatment can be used as a reference RTC profile for comparison to RTCs obtained from the patient after treatment. A reference profile can also be generated from a cancer cell line or a cancer biopsy cell.

The response of the RTCs to one or more therapeutic agents and/or therapeutic modality can be analyzed. The RTCs can be expanded in vitro or ex vivo in three-dimensional culture prior to testing the response of the RTCs to the therapeutic agents and/or therapeutic modalities. RTCs (an individual cell or a cluster of RTCs) can be expanded to about 50, 100, 500, 1,000, 5,000, 10,000 cells or more. In some aspects, RTCs can be responsive to the therapeutic agents and/or therapeutic modalities. The RTCs can be considered responsive if the therapeutic agents and/or therapeutic modality results in a decrease in the number, proliferation rate, viability of cells, and/or another suitable parameter of cell viability. In some aspects, the RTCs are considered responsive if the therapeutic agents and/or therapeutic modalities decrease cell number, proliferation rate, and/or viability by at least 50%, 60%, 70%, 80%, 90%, 95% or 100%, or by 50-60%, 55-65%, 60-70%, 65-75%, 70-80%, 75-85%, 80-90%, 85-95%, or 90-100%. In some aspects, RTCs can be considered non-responsive to therapeutic agents and/or therapeutic modalities if the therapeutic agent or modality does not decrease in the number, proliferation rate and/or viability of cells by more than 5%, 10%, 15%, 20%, 30%, 40%, or up to 50%. In some aspects, only a portion of the RTCs cultured in 3D cell culture are utilized for testing with the therapeutic agents and/or therapeutic modalities.

In some aspects, the methods include conducting molecular profiling on the RTCs. In some aspects, molecular profiling can be performed on RTCs prior to expansion in 3D cell culture. In some aspects, molecular profiling is performed after expansion in 3D culture. In some aspects, molecular profiling is performed after treatment with the therapeutic agents and/or therapeutic modalities. The reference RTC profile library can include the molecular profiles of more than one RTC obtained from at least one subject. For examples the reference profile library can include molecular profiles of 2, 3, 4, 5, 6, 7, 8, 9, 10, 15, 20, 25, 30, 35, 40, 45, 50, 100, 200, 300 or more RTCs. In some aspects, the preparation of the reference RTC profile library can further include obtaining the morphological profile of the RTCs that are responsive and RTCs that are not responsive to the therapeutic agents or modalities.

The present disclosure also provides methods of determining treatment protocol for a cancer patient. The method can include isolating and enriching RTCs from a biological sample of the cancer patient. The molecular profiling of the RTCs can then be conducted to establish a molecular profile. A molecular profile of the RTCs of the cancer patient (e.g., an animal, mammal, or human patient) can then be compared to the reference RTC profile library. Comparison of the molecular profile of the RTCs of the cancer patient with reference RTC profile library can be used to identify one or more reference RTC profiles that are similar the molecular profile of the RTCs of the cancer patient. A score can be assigned based on the similarity of the profiles. In some aspects, a treatment protocol can be identified based on the similarity of the molecular profile of the RTCs of the cancer patient to reference RTC profile library members.

In some aspects, the molecular profile of RTCs of the cancer patient can also be compared to the molecular profile of reference cancer cells, which are also optionally grown in 3D culture. The reference cancer cells can be a cancer cell line, a cancer biopsy, and/or a patient derived xenograft. In some aspects, the molecular profile of the reference cancer cells can be obtained prior to and/or after treatment with a therapeutic agent and/or a therapeutic modality.

### Biological Samples

In various aspects, the RTCs can be isolated from a biological sample from a subject. In some aspects, the biological sample can be a liquid biopsy of the cancer (e.g., a blood sample enriched for tumor cells). In some aspects, the biological sample can be a cheek swab sample; a mucus sample; whole blood sample, a blood sample, a serum sample; a plasma sample; a urine sample; a saliva sample; a semen sample; lymphatic fluid sample; a fecal sample, a sputum sample, a cerebrospinal fluid sample, an interstitial fluid sample, a peritoneal fluid sample, a pleural fluid sample, a cyst fluid sample, a semen sample, a mucous sample, an intestinal fluid sample, or any other body fluid or biofluid; a cell sample; a tissue sample; a tumor sample; tumor biopsy, or their combinations.

In some aspects, a biological sample can be any fluid derived from or in association with epithelial tissue. Examples include, but are not limited to, whole blood, pleural effusion, pericardial effusion, ascites, CSF, urine, fluid from ductal lavage, fluid from bronchoalveolar lavage, fluid from endoscopic retrograde cholangiopancreatography (ERCP) duct cytologic brushing, fluid from esophageal brushing, fluid from cervical brushing, fluid from uterine brushing, and fluid from cystic lesions that can be aspirated with a needle.

In some aspects, a biological sample can be a fine-needle aspirated tissue sample or bone marrow sample.

In some aspects, a biological sample can be obtained from a patient who has been previously treated with a therapeutic agent or a therapeutic modality.

In some aspects, RTCs can be obtained from two or more biological samples of the same subject.

### Reference Cancer Cell

In some aspects, the profile of one or more RTCs (e.g., a single RTC or a cluster of RTCs can be compared to a reference cancer cell. As used herein, a reference cancer cell can be any cancer cell obtained from a tumor from a cancer patient or a cell into which oncogenic mutations have been introduced. A reference cancer profile can comprise a profile from a single cancer cell, a cluster of cancer cells, or a combined profile from several cancer cells (e.g. profiles of cancer cells obtained from cancer patients, profiles of cancer cells from cancer patients having the same type of cancer, profiles of cancer cells from cancer patients who have had treatment for cancer, profiles of cancer cell lines derived from the same type of cancer). The tumor can be a primary tumor or a metastatic tumor. The reference cancer cell can be a cancer cell line established by the serial culture of cancer cells in a laboratory. The reference cancer cell can also be obtained from a biopsy of the patient tumor. In some aspects, the reference cancer cells can be obtained from patient-derived xenografts (PDX). In PDX, freshly resected tumor pieces are implanted by sub cutaneous or orthotopic injections into immunocompromised mice.

### Isolation and Enrichment of RTCs

RTCs can be isolated from one or more biological samples from a patient described herein.

RTCs can be isolated from a subject using device that preferentially captures RTCs. RTCs can be isolated and/or enriched using microdevices and/or microfluidic devices. For examples, RTCs can be isolated using a biological fluid filtration system that includes a scanner which uses digital holographic microscopy to produce a holographic image of the cells as they move through the microfluidic device. See WO2021155322; US Publ. No. 2023/004052, which are incorporated herein by reference. Based on the analysis of the holographic images of the cells, RTCs can be selected for isolation into a separate reservoir that keeps the RTCs separate from the other cells in a biological sample.

RTCs can be enriched by relying on one or more features of the RTCs that can differentiate the RTCs from other cells. Cells that are not RTCs are herein referred to as “non-RTCs.” In one aspect, the non-RTCs can be blood cells e.g., red blood cells (RBCs) or white blood cells (WBCs), endothelial cells, and/or stromal cells. RTCs can be enriched utilizing a physical enrichment method and/or a biological enrichment method. Multiple enrichment methods can be applied to a biological sample. The multiple methods can be performed in parallel or in succession.

Physical enrichment methods can involve enrichment of RTCs based on density, size, deformability, and/or electric charge.

In some aspects, RTCs can be enriched by density-based centrifugation. Several commercially available separation media can be used for enrichment of RTCs including, but not limited to, Ficoll-Paque (GE Healthcare, Chicago, IL, USA), Percoll (GE Healthcare), Lymphoprep (STEMCELL Technologies, Vancouver, Canada), and/or OncoQuick (Greiner Bio-One, Kremsmunster, Austria). In some aspects, the RTCs can be enriched by sedimentation.

In some aspects, the RTCs can be enriched based on size. For example, when the RTCs are isolated from a blood sample, the differential size of hematological and non-hematological cells (such as the RTCs) can be used to enrich RTCs. Size-based enrichment of RTCs can include isolation by the size of epithelial tumor (ISET) cells and microelectromechanical system (MEMS)-based micro-filter means. ISET is a size-based method to capture RTCs by directly filtering the biological sample (e.g., blood) through a calibrated, polycarbonate Track-Etch-type membrane with 8-μm-diameter pores.

Isolation and/or enrichment of RTCs can involve inertial microfluidic technology which applies the effects of the secondary flow of the macroscopic fluid to the microscopic flow channel and thus can separate RTCs from other cell types based on their morphological differences.

Enrichment of RTCs can also include inertial microfluidics-based separation techniques termed Dean flow fractionation (DEF), which is a spiral RTC enrichment chip that separates cells based on size; smaller non-RTCs (e.g., blood cells) can migrate along Dean vortices towards the inner wall and then back to the outer wall again, whereas larger RTCs experience additional strong inertial lift forces and collect along the microchannel inner wall.

In some aspects, RTCs can be enriched based on another physical property of deformability. In some aspects, RTCs can be more deformable than non-RTCs and can be enriched by measuring the time required for RTCs versus non-RTCs to go through a microfluidic device. A device containing a parallel network of fluidic channels with capture chambers can be utilized. Larger RTCs can be captured and retained in the chambers, whereas smaller non-RTCs (e.g., blood cells) are not retained in the fluidic channels.

In some aspects, RTCs can also be separated by di-electrophoresis (DEP) as RTCs can exhibit a distinctive dielectric property. Enrichment based on differences in surface charge and polarizability minimize the injury of captured RTCs, which is favorable to subsequent culture of the enriched RTCs.

In some aspects, RTCs can also be enriched based on their biological properties. e.g., the presence of cell surface adhesion molecules or the absence of hematological surface antigen markers.

In some aspects, RTCs are derived from tumors of epithelial origin which express proteins on the cell membrane that help maintain cell-cell junction and adhesion, epithelial cell-specific antigens. These proteins can be utilized as “markers” to positively enrich for RTCs from non-RTCs using antibodies directed to the markers. Non-limiting examples of markers that can be used for enrichment of include, EpCAM, CK8, CK18 and CK19. In some embodiments, RTCs can be enriched using antibodies to markers for epithelial mesenchymal transition e.g., N-cadherin or vimentin. RTCs can also be enriched using antibodies to tumor-specific markers proteins e.g., CEA, EphB4, EGFR, PSA, HER-2, MUC-1. Separation based on antibodies to cell surface proteins can be carried out using magnetic bead separation, where antibody-labeled ferroparticles capture RTCs in a magnetic field or using non-magnetic methods using microfluidic chips.

RTCs can also be enriched by depletion of non-RTCs. In some aspects, non-RTCs can be hematopoietic cells e.g., peripheral blood mononuclear cells (PBMC). In one aspects, the non-RTCs can be red blood cells. Red blood cells can be removed from a sample by the inclusion of an RBC lysis step. RBCs can be lysed by applying a lysis buffer that preferentially lyses RBC. The lysis buffer can include NH₄Cl, and/or KHCO₃.

In some aspects, RTCs can be enriched by applying digital holographic microscopy to cells flowing in a microchannel. See WO2021155322; US Publ. No. 2023/004052, which are incorporated herein by reference. The resulting interference patterns or holograms can then be computationally reconstructed to obtain 3D positional information, size and intensity features that can be used to differentiate RTCs from non RTCs.

RTCs can be isolated and/or enriched using microdevices and/or microfluidic devices. For examples, RTCs can be isolated and/or enriched using a biological fluid filtration system. U.S. Patent Publication US20210237064A1, which includes a receiving device for receiving a biological sample. The system can include an inlet, and an outlet. A second outlet can be connected to the receiving device. The biological sample is scanned by a scanner to produce data related to the biological sample. The valve of the system can be controlled to direct the RTCs in the biological sample to a particular output based on the scanning data. In some aspects, the scanner can utilize digital holographic microscopy to produce a holographic image of the cells. The systems can include a multi-stage separation methodology with a first step including passive inertial sorting with or without the use of buffer fluids.

From the enriched RTC samples, single cells or RTC clusters (e.g., clusters of 2, 5, 10, 20 or more RTCs) can be isolated using the devices described herein e.g., the microdevices and/or microfluidic devices described herein. In some aspects, single RTCs or clusters of RTCs can be isolated using digital holographic microscopy. RTCs can be isolated into a separate reservoir that keeps the RTCs separate from the other cells in a biological sample.

### Three-Dimensional (3D) Culture

RTCs exist in a small fraction among vast numbers of cell types. Expanding RTCs ex vivo can be conducted for reproducible examination of their molecular composition and behaviors in vitro, ex vivo, or in vivo.

In some aspects, RTCs can be expanded ex vivo or in vitro in three-dimensional (3D) cell culture. In 3D cell culture, RTCs are cultured with surrounding extracellular framework in three dimensions. RTCs can be grown with or without a supporting scaffold. In some aspects, RTCs can be cultured in a scaffold-free 3D cell culture. RTCs can be cultured in cell culture surfaces coated with hydrophilic polymer to prevent attachment of cells to cell culture surface. Cell culture surfaces can be patterned or can incorporate microwells. In one aspect, RTCs can be cultured in a suspended drop of a medium, allowing cells to aggregate and form spheroids.

In 3D cell culture, RTCs can be grown as organoids, spheroids, microtumors, or ex vivo tumors. Organoids are self-organized three-dimensional tissue cultures developed from RTCs. Spheroids are self-assembling RTC aggregates that grow so as to prevent attachment to a flat surface. Microtumors include RTCs supported in 3D extracellular matrix (ECM) and comprising perfused microvessels. Ex vivo tumors include 3D monolayer cultures from RTCs and 3D cultures developed from RTCs.

RTCs can be cultured as spheroids. In spheroid culture, RTCs grow as an aggregation of cells into a round cell cluster that is a three-dimensional structure. RTC spheroids can be round and uniform in shape. RTCs can be cultured as organoids. As used herein, the term organoid refers to an artificial organ model of live cells in a three-dimensional or multi-layered configuration and can include cells other than RTCs. In some aspects, organoids can include an ordered structure.

RTCs can be cultured as microtumors prepared as RTC spheroids, endothelial cell tubules, and stromal cells cultured in an extracellular matrix. The extracellular matrix can include, e.g., collagens, proteoglycans (such as heparan sulphate proteoglycans, versican and hyaluronan) and glycoproteins (such as laminins, elastin, fibronectin and tenascins). An ECM can comprise an interstitial matrix and a basement membrane. The interstitial matrix forms porous three-dimensional networks around cells that interconnect cells in the stroma and can connect to the basement membrane. An interstitial matrix provides structural integrity and modulates processes such as cell differentiation and migration. An interstitial matrix can be comprised of collagens I, III, V, etc., fibronectin and elastin. RTCs can remodel the interstitial ECM with biophysical and biochemical changes, which can affect cell signaling, ECM stiffness, cell migration, and tumor progression.

The basement membrane is a stable, sheet-like, dense structure that can comprise collagen IV and laminins, which are interconnected through different network-bridging proteins such as nidogen and heparan sulphate proteoglycans (HSPGs). RTCs can remodel the basement membrane to invade stromal tissue and become a malignant tumor.

In some aspects, RTCs can be cultured with a scaffold such as, but not limited to, hydrogels or inert matrices.

RTCs can be cultured in a microenvironment that mimics at least in part a cellular niche in which the tumors from the RTCs are derived or naturally reside. RTCs can be cultured in the presence of biomaterials or synthetic materials that provide interactions with cellular membrane proteins such as integrins. In some aspects, the RTCs can be cultured in a 3D matrix that includes, polysaccharides, elastin, and/or glycoproteins, (e.g., collagen, entactin (nidogen), fibronectin, and/or laminin). In some aspects, RTCs can be cultured in 3D cell culture using Matrigel®, collagen, alginate, agarose, QGel® Matrix, Engelbreth-Holm-Swarm (EHS) mouse sarcoma cell-based preparations (e.g., Cultrex® Basement Membrane Extract (Trevigen, Inc.) or Matrigel™ (BD Biosciences)).

For 3D culture, the media for RTC culture can include factors such as, but not limited to, A83-01, B27 supplement (with or without Vitamin A), cAMP, Dihydrotestosterone (DHT), EGF, FGF, Forskolin, Gastrin, Glucocorticoids, Glutamax, GSK3b inhibitor, GSK3a inhibitor (CHIR99021), Heparin, Hepatocyte growth factor, HEPES, IGF, insulin, insulin-like growth factor (IGF), I-glutamine, N2 supplement, N-Acetylcysteine, NEAA, Nicotinamide, Noggin, Non-essential amino acid (NEAA), p38 inhibitor, p38 inhibitor (SB202190), PGE2, retinoic acid, RHOK (Y-27632), ROCK inhibitor, R-spondin 1, SB202190, TGFβ inhibitor (A-83-01), and/or WNT.

RTCs can be cultured in a culture medium, which, as used herein, refers to a solid or a liquid substance used to support the growth of cells (isolated cells or cells or within a tissue or an organoid). The culture medium used herein can be a water-based medium which includes a combination of substances such as salts, nutrients, minerals, vitamins, amino acids, nucleic acids, proteins such as cytokines, growth factors and hormones, all of which are needed for cell growth and maintenance of cells metabolically active. For example, a culture medium can be a synthetic tissue culture medium such as mTESR, E8, NutriStem, PluriSTEM, William's E, RPMI 1640, DMEM (Dulbecco's Modified Eagle Medium), MEM (Minimum Essential Medium Eagle), KO-DMEM (Knockout Dulbecco's Modified Eagle Medium), IMDM (Iscove's Modified Dulbecco's Medium), Neurobasal medium, EGM-2 (Endothelial growth medium), EMV-2 (Endothelial microvascular medium), HMM (Hepatocytes Maintenance Medium), Medium 199 or MCDB105/153/131/170.

RTCs can be cultured with one or more cells that are not RTCs. In some aspects, the RTCs can be cultured with immune cells. Any immune cell that can be incorporated into a co-culture is suitable for use with methods of the disclosure. Non-limiting examples of immune cells for co-culture with RTCs include, intra-epithelial lymphocytes (IELs), tumor infiltrating lymphocytes (TILs), peripheral blood mononuclear cells (PBMCs), peripheral blood lymphocytes (PBLs), T cells, cytotoxic T lymphocytes (CTLs), B cells, NK cells, mononuclear phagocytes, alpha/beta receptor T-cells and gamma/delta receptor T cells, and/or myeloid-derived suppressor cells. Immune cells can be allogeneic with the RTCs. In some aspects, the RTCs and the immune cells can be obtained from the same subject. In some aspects, the immune cells can be HLA-matched with the RTCs.

### Profiling Released Tumor Cells

Characterization of RTCs by profiling can serve as a surrogate for RTC biological activity underlying response to a therapeutic agent and/or a therapeutic modality. As used herein, “profiling”, refers to the measurement of at least one feature or property of a cell such as, but not limited to, a molecular feature (e.g., protein expression profile, mRNA expression profile), or a morphological feature.

In some aspects, the profiling can be molecular profiling, morphological profiling, and/or holographic profiling.

### Molecular Profiling

RTC profiling can include analysis of molecular features such as, DNA, RNA, mRNA, protein, enzymatic activity, and/or metabolites of the cells.

Molecular profiling of the RTCs can be conducted after isolation/enrichment from a biological sample, before or after expansion of RTCs in 3D cell culture, and/or before or after treatment of the RTCs with at least one therapeutic agent and/or a therapeutic modality. Molecular profiling can also be applied to reference cancer cells described herein.

Molecular profiling can be genomic profiling, transcriptomic profiling and/or proteomic profiling. Molecular profiling can be single cell profiling, wherein each RTC or RTC cluster is sequenced individually to obtain capture the potential heterogeneity among the RTCs of a biological sample. In some aspects, all the RTCs from a biological sample can be profiled together through an approach referred to herein as the “bulk” approach. The bulk profiling approach can provide average profile information for a population of RTCs.

Nucleic acid or protein samples for molecular profiling can be derived from RTCs using methods described below.

Genomic profiling of RTCs can be applied to study structural changes to the DNA sequence that can occur due to treatment with one or more therapeutic agents and/or therapeutic modalities. Genomic profiling can be performed using next generation sequencing methods. Non-limiting examples of next generation sequencing technologies can include ion Torrent, Illumina, SOLID; massively parallel signature sequencing solid phase, reversible staining-terminator sequencing; single-molecule real-time sequencing, Pyrosequencing, Nanopore Sequencing, GenapSys Sequencing, and DNA nanosphere sequencing.

Transcriptomic profiling can involve measurement of mRNA expression. mRNA expression can detected by, for example, but is not limited to: PCR procedures, RT-PCR, quantitative PCR or RT-PCR, Northern blot analysis, differential gene expression, RNA protection assays, microarray analysis, hybridization methods, next generation sequencing, and the like. Non-limiting examples of next generation sequencing technologies can include ion Torrent, Illumina, SOLID; massively parallel signature sequencing solid phase, and reversible staining-terminator sequencing.

Proteomic analysis can be used to study the expression of the proteins in RTCs before and after treatment with a therapeutic agent and/or a therapeutic modality. Proteomic profiling can reveal activated pathways, surface antigen expression, and related targets. For proteomic analysis, the 2-D electrophoresis method is applied to isolate the proteins from the gel. The proteins can be digested into peptides that mass spectrometry (MS) can identify. In some aspects, a “shotgun” proteomics approach can be used in which the digestion of proteins occurs without fractionation, and liquid chromatography is used to separate the peptides identified by MS. The molecular mass of proteins can be calculated by using electrospray ionization (ESI) followed by matrix-assisted laser desorption/ionization (MALDI). Proteomic analysis can be used to identify differential protein expression and/or changes in post translational modifications of the proteins in RTC samples.

### Morphological Profiling

RTCs that are treated with one or more therapeutic agents or therapeutic modalities can exhibit different morphological features compared to RTCs that are not treated with therapeutic agent and/or modality. RTC expression profiles can be compared to reference cell profiles (e.g., RTCs not treated or control cells that are treated or not treated) before or after treatment with one or more therapeutic agents or therapeutic modalities. RTCs that are sensitive to one or more therapeutic agents and/or modalities can also exhibit different morphological features compared to RTCs that are resistant to one or more therapeutic agents and/or modalities. In some aspects, the comparison of morphological profiles can be used to evaluate the response of a sample to a therapeutic agent and/or modality. In some aspects, comparison of the morphological profiles can be used to differentiate responses of the RTCs after the RTCs have been contacted with a different therapeutic agent and/or modality. Morphological profiles can also be used to determine the degree of cell death, cell stress, or damage in an RTC or a population of RTCs.

In some aspects, RTC profiling can include analysis of morphological features such as cell shape, size, area, volume, texture, thickness, roundness, structure, form, aspect ratio, geometry or any other physical attributes of the cell. In some aspects, morphological profile, can include cell behavioral phenotypes e.g., motility.

In some aspects, morphological profiling can include an image based assay of the sub compartments, such as organelles (e.g., nucleus, Golgi apparatus, mitochondria, endoplasmic reticulum, lysosomes), macromolecules, membranes, or metabolites, can be specifically labelled in the RTCs in the fixed, or living state and detected by a digital imaging system, for example, such as an automated fluorescence imaging system, using, for example, either higher resolution wide-field, confocal, light sheet, or super-resolution fluorescence microscopy, or automated, high-content screening systems. Observations can be carried out in situ (that is, on either living, or fixed cells), without extracting material from the cell. The cells can also be lysed and extracted material obtained for analysis. In morphological profiling, measured features can include staining intensities, textural patterns, size, and shape of the labeled cellular structures, as well as correlations between stains across channels, and adjacency relationships between cells and among intracellular structures.

Each RTC sample analyzed can be assigned a score based on one or more morphological characteristics. In some aspects, a machine learning algorithm can be configured to extract one or more morphological features from the RTCs described herein.

### Holographic Profiling

In some aspects, a holographic image of an RTC can be prepared as part of holographic profiling. As used herein, a “holographic image” refers to a three-dimensional image formed by the interference of light beams from a laser or other coherent light sources. See WO2021155322; US Publ. No. 2023/004052. Using holographic imaging systems, a 3D morphological signature of the individual RTCs following through a microchannel can be captured. Algorithms can be utilized to identify cells based on the acquired data from the RTCs e.g., neural network type classifiers to decision trees.

Holographic profiling can be conducted using digital holographic microscopy. Digital holography can be used to record a wave front diffracted from an object by a light source (e.g., monochromatic laser). Utilizing the interference of light from the light source, both amplitude and phase information of an object wave can be recorded to produce a hologram containing the information of the object wave. A three-dimensional image can then be reconstructed from the hologram by a control unit. Various types of digital holography can be utilized with the methods described herein, including but not limited to off-axis Fresnel, Fourier, image plane, in-line, Gabor, and phase-shifting digital holography

In some aspects, a two-dimensional “hologram” of a cell can be generated by superimposing the multiple images of the individual cell. The “hologram” can be analyzed to automatically classify characteristics of the cell based upon features including but not limited to the morphological features of the cell.

Digital holographic microscopy can be utilized to observe living cells within a fluid. From the recorded interference pattern of living cells, the intensity and phase shift across various points of the cells can be numerically computed by a control unit. The control unit can measure phase delay images of RTCs within the biological samples to provide quantitative information about the morphological properties (e.g., cellular dry mass, surface texture, shape, etc.) of individual cells within the fluid. In some aspects, the systems and methods described herein can utilize these quantitative indicators of morphological properties in an algorithm to distinguish between cell types within the biological sample. By way of example and without limitation, the quantitative indicators can be cell thickness, cell area, cell volume, cell dry mass, the phase shift across the cell, surface roughness and texture, cell shape, elongation, convexity, luminance, circularity, solidity, and the like. Each RTC sample analyzed can be assigned a score based on one or more optical and/or holographic characteristics.

### Treatment Protocol

The RTCs obtained by the methods described herein can be representative of cancer cells within a patient. In some aspects, the RTCs e.g., the CTCs described herein can be associated with the tumorigenic and/or metastatic potential of cancer cells. Thus, the RTCs described herein can be used to determine a treatment protocol for a patient based on the sensitivity of a subject's RTCs to a therapeutic agent and/or therapeutic modality. The treatment protocol for a subject can include one or therapeutic agents which can be combined with one or more treatment modalities. In some aspects, the treatment protocol can be determined by comparing the profile of the RTCs of the cancer patient to the reference RTC profile library because the susceptibility of the RTCs in the profile library are known after the initial analysis. The comparison of RTCs of a subject to the RTC profile library can guide a medical professional to a treatment or therapeutic modality based on similar RTCs from the library. Multiple biological samples of a patient can be used to isolate and/or enrich RTCs. More than one type of RTC (e.g., RTCs originating from different tumors or areas in the body) can be analyzed in a subject (e.g., about 1, 2, 3, 4, 5 or more). RTCs can be isolated and/or enriched from the cancer patient at various timepoints and their profiles can be compared to the reference RTC library and/or to earlier profiles from the patient. The various timepoints for RTC sampling in a cancer patient can include prior to administration of a treatment protocol, during the administration of a treatment protocol and/or after a treatment protocol is provided. In some aspects, any change in the molecular, holographic, and/or morphological profile of RTCs can be used to prepare a new treatment protocol or modify an existing treatment protocol.

In some aspects, the treatment protocol can be applied to a cancer, such as, but not limited to, tumors of the gastrointestinal tract (colon carcinoma, rectal carcinoma, colorectal carcinoma, colorectal cancer, colorectal adenoma, hereditary nonpolyposis type 1, hereditary nonpolyposis type 2, hereditary nonpolyposis type 3, hereditary nonpolyposis type 6; colorectal cancer, hereditary nonpolyposis type 7, small and/or large bowel carcinoma, esophageal carcinoma, tylosis with esophageal cancer, stomach carcinoma, pancreatic carcinoma, pancreatic endocrine tumors), endometrial carcinoma, dermatofibrosarcoma protuberans, gallbladder carcinoma, Biliary tract tumors, prostate cancer, prostate adenocarcinoma, renal cancer (e.g., Wilms' tumor type 2 or type 1), liver cancer (e.g., hepatoblastoma, hepatocellular carcinoma, hepatocellular cancer), bladder cancer, embryonal rhabdomyosarcoma, germ cell tumor, trophoblastic tumor, testicular germ cells tumor, immature teratoma of ovary, uterine, epithelial ovarian, sacrococcygeal tumor, choriocarcinoma, placental site trophoblastic tumor, epithelial adult tumor, ovarian carcinoma, serous ovarian cancer, ovarian sex cord tumors, cervical carcinoma, uterine cervix carcinoma, small-cell and non-small cell lung carcinoma, nasopharyngeal, breast carcinoma (e.g., ductal breast cancer, invasive intraductal breast cancer, sporadic; breast cancer, susceptibility to breast cancer, type 4 breast cancer, breast cancer-1, breast cancer-3; breast-ovarian cancer), squamous cell carcinoma (e.g., in head and neck), neurogenic tumor, astrocytoma, ganglioblastoma, neuroblastoma, lymphomas (e.g., Hodgkin's disease, non-Hodgkin's lymphoma, B cell, Burkitt, cutaneous T cell, histiocytic, lymphoblastic, T cell, thymic), gliomas, adenocarcinoma, adrenal tumor, hereditary adrenocortical carcinoma, brain malignancy (tumor), various other carcinomas (e.g., bronchogenic large cell, ductal, Ehrlich-Lettre ascites, epidermoid, large cell, Lewis lung, medullary, mucoepidermoid, oat cell, small cell, spindle cell, spinocellular, transitional cell, undifferentiated, carcinosarcoma, choriocarcinoma, cystadenocarcinoma), ependymoblastoma, epithelioma, erythroleukemia (e.g., Friend, lymphoblast), fibrosarcoma, giant cell tumor, glial tumor, glioblastoma (e.g., multiforme, astrocytoma), glioma hepatoma, heterohybridoma, heteromyeloma, histiocytoma, hybridoma (e.g., B cell), hypernephroma, insulinoma, islet tumor, keratoma, leiomyoblastoma, leiomyosarcoma, leukemia (e.g., acute lymphatic, acute lymphoblastic, acute lymphoblastic pre-B cell, acute lymphoblastic T cell leukemia, acute-megakaryoblastic, monocytic, acute myelogenous, acute myeloid, acute myeloid with eosinophilia, B cell, basophilic, chronic myeloid, chronic, B cell, eosinophilic, Friend, granulocytic or myelocytic, hairy cell, lymphocytic, megakaryoblastic, monocytic, monocytic-macrophage, myeloblastic, myeloid, myelomonocytic, plasma cell, pre-B cell, promyelocytic, subacute, T cell, lymphoid neoplasm, predisposition to myeloid malignancy, acute nonlymphocytic leukemia), lymphosarcoma, melanoma, mammary tumor, mastocytoma, medulloblastoma, mesothelioma, metastatic tumor, monocyte tumor, multiple myeloma, myelodysplastic syndrome, myeloma, nephroblastoma, nervous tissue glial tumor, nervous tissue neuronal tumor, neurinoma, neuroblastoma, oligodendroglioma, osteochondroma, osteomyeloma, osteosarcoma (e.g., Ewing's), papilloma, transitional cell, pheochromocytoma, pituitary tumor (invasive), plasmacytoma, retinoblastoma, rhabdomyosarcoma, sarcoma (e.g., Ewing's, histiocytic cell, Jensen, osteogenic, reticulum cell), schwannoma, subcutaneous tumor, teratocarcinoma (e.g., pluripotent), teratoma, testicular tumor, thymoma and trichoepithelioma, gastric cancer, fibrosarcoma, glioblastoma multiforme; multiple glomus tumors, Li-Fraumeni syndrome, liposarcoma, lynch cancer family syndrome II, male germ cell tumor, mast cell leukemia, medullary thyroid, multiple meningioma, endocrine neoplasia myxosarcoma, paraganglioma, familial nonchromaffin, pilomatricoma, papillary, familial and sporadic, rhabdoid predisposition syndrome, familial, rhabdoid tumors, soft tissue sarcoma, and Turcot syndrome with glioblastoma.

RTCs of the disclosure (e.g., RTCs before or after expansion to 3D culture) can be treated with one or more therapeutic agents and/or modalities. A treatment protocol as described herein, can include one or therapeutic agents or modalities

### Therapeutic Agents

In some aspects, the therapeutic agent can be a targeted therapeutic agent, a chemotherapeutic agent, a hormone therapy agent, and/or an immunotherapeutic agent.

In some aspects, a targeted therapeutic agent refers to an agent that can target nucleic acids or proteins that that control growth, proliferation and/or the spread of cancer cells. The therapeutic agents can be small molecule drugs, antibodies, and/or recombinant nucleic acids.

Non-limiting examples of therapeutic agents that are small molecule include, SUV4-20 (SUV420H1 or SUV420H2), a tyrosine kinase inhibitor, a retinoid-like compound, a weel kinase inhibitor, an anaplastic lymphoma kinase inhibitor, an aurora A kinase inhibitor, an aurora B kinase inhibitor, a reversible inhibitor of eukaryotic nuclear DNA replication, an antimetabolite antineoplastic agent, an ataxia telangiectasia and Rad3-related protein (ATR) kinase inhibitor, an ATM kinase inhibitor, a checkpoint kinase inhibitor, a GSK-3a/b inhibitor, a proteasome inhibitor, an AXL or RET inhibitor, a c-Met or VEGFR2 inhibitor, an alkylating antineoplastic agent, a DNA-PK and/or m TOR inhibitor, an inhibitor of mammalian target of rapamycin (mTOR), a checkpoint kinase 1 (CHK1) inhibitor, a retinoic acid receptor b (RARP) or RARy antagonist, a retinoic acid receptor (RAR)y-selective agonist, RARy-selective retinoid, inducer of apoptosis, CDK2 a RAR agonist, a chemotherapy, a tyrosine kinase inhibitor antineoplastic agent, an antimicrotubular antineoplastic agent, a topoisomerase inhibitor antineoplastic agent, a sodium-glucose cotransporter-2/SGLT2 inhibitor, an inhibitor of the tropomyosin receptor kinases A, B and C, C-ros oncogene 1 and anaplastic lymphoma kinase, a topoisomerase inhibitor antineoplastic agent, an inhibitor of mTOR, an inhibitor of phosphatidylinositol 3-kinase (PI3K), an inhibitor of RIP3K, an analog of cyclophosphamide, an SGLT2 inhibitor, aWnt/p-catenin inhibitor, a tyrosine kinase inhibitor that interrupts the HER2/neu and epidermal growth factor receptor/EGFR pathways, an inhibitor of tropomyosin kinase receptors TrkA, TrkB, and TrkC, a cyclin-dependent kinase (CDK) inhibitor, a CDK7 inhibitor, an inhibitor of VEGFR1, VEGFR2 and VEGFR3 kinases, a DNA-PK/PI3K/mTOR inhibitor, a poly ADP ribose polymerase (PARP) inhibitor, an inhibitor of Rac GTPase, a taxane, a Bromodomain And PHD Finger Containing 1 (BRPFI) bromodomain inhibitor, a mitogen-activated protein kinase-activated protein kinase 2 (MAPK2) inhibitor, a RAF inhibitor, a histone deacetylase (HD AC) inhibitor, a CDK1 inhibitor, aTGF-beta/Smad inhibitor, a Pim kinase inhibitor, a DNA topoisomerase I inhibitor, active metabolite of CPT-11/Irinotecan, an atypical retinoid, apoptosis inducer, a multi-kinase inhibitor, a fms-like tyrosine kinase-3 (FLT3) inhibitor, a MEK inhibitor, an inhibitor of extracellular signal-regulated kinase (ERK) 1 and/or 2, or a DNA-dependent protein kinase/DNA-PK inhibitor.

In some aspects, the therapeutic agent can be a biological agent e.g., an antibody. The antibody can be a monoclonal antibody or a polyclonal antibody. In some aspects, the therapeutic antibody can be Trastuzumab duocarmazine, Glofitamab, Mirikizumab, Mirvetuximab soravtansine, Nirsevimab, Tremelimumab, Donanemab, Lecanemab, Tislelizumab, Penpulimab, Sintilimab, Teplizumab, Toripalimab, Omburtamab, Retifanlimab, Ublituximab, Inolimomab, Oportuzumab monatox, Narsoplimab, Spesolimab, Teclistamab, Mosunetuzumab, Tixagevimab, cilgavimab, Relatlimab, Tebentafusp, Faricimab, Sutimlimab, Sotrovimab, Regdanvimab, Casirivimab +imdevimab, Tezepelumab, Tisotumab vedotin, Amivantamab, Anifrolumab, Loncastuximab tesirine, Bimekizumab, Tralokinumab, Evinacumab, Aducanumab, Dostarlimab, Ansuvimab, Margetuximab, Naxitamab, Atoltivimab, Maftivimab, and Odesivimab-ebgn, Belantamab mafodotin, Tafasitamab, Satralizumab, Inebilizumab, Sacituzumab govitecan, Teprotumumab, Isatuximab, Eptinezumab, [fam]-trastuzumab deruxtecan, Enfortumab vedotin, Crizanlizumab, Brolucizumab, Polatuzumab vedotin, Risankizumab, Romosozumab, Caplacizumab, Ravulizumab, Emapalumab, Cemiplimab, Fremanezumab, Moxetumomab pasudotox, Galcanezumab, Lanadelumab, Mogamulizumab, Erenumab, Tildrakizumab, Ibalizumab, Burosumab, Durvalumab, Emicizumab, Benralizumab, Ocrelizumab, Guselkumab, Inotuzumab, Sarilumab, Dupilumab, Avelumab, Brodalumab, Atezolizumab, Bezlotoxumab, Olaratumab, Reslizumab, Obiltoxaximab, Ixekizumab, Daratumumab, Elotuzumab, Necitumumab, Idarucizumab, Alirocumab, Mepolizumab, Evolocumab, Dinutuximab, Secukinumab, Nivolumab, Blinatumomab, Pembrolizumab, Ramucirumab, Vedolizumab, Siltuximab, Obinutuzumab, Ado-trastuzumab emtansine, Raxibacumab, Pertuzumab, Brentuximab vedotin, Belimumab, Ipilimumab, Denosumab, Tocilizumab, Ofatumumab, Canakinumab, Golimumab, Ustekinumab, Certolizumab pegol, Catumaxomab, Eculizumab, Ranibizumab, Panitumumab, Natalizumab, Bevacizumab, Cetuximab, Efalizumab, Omalizumab, Tositumomab-I131, Ibritumomab tiuxetan, Adalimumab, Alemtuzumab, Gemtuzumab, ozogamicin, Trastuzumab, Infliximab, Palivizumab, Basiliximab, Daclizumab, Rituximab, Abciximab, Edrecolomab, Nebacumab, and/or Muromonab-CD3.

In some aspects, the therapeutic agent can be a nucleic acid molecule targeting expression of one or more genes of the RTCs. The nucleic acid molecule can be an RNA interference molecule e.g., a microRNA or a small interfering RNA.

In some aspects, the therapeutic agent can be a hormone therapy agent, which, as used herein, can refer to an agent that can target one or more cellular pathways in cancer cells that are mediated by hormones. In some aspects, the therapeutic agent can be tamoxifen, toremifene, fulvestrant, Anastrozole, Exemestane, and/or Letrozole.

In some aspects, the therapeutic agent can be an immunotherapeutic agent. An immunotherapeutic agent can be an antibody and fragments and variants thereof, a cancer specific T cell receptor (TCR) and variants thereof, an anti-tumor specific chimeric antigen receptor (CAR), a chimeric switch receptor, an inhibitor of a co-inhibitory receptor or ligand, an agonist of a co-stimulatory receptor and ligand, a cytokine, chemokine, a cytokine receptor, a chemokine receptor, a soluble growth factor, a metabolic factor, a suicide gene, a homing receptor, or any agent that induces an immune response in a cell and/or a subject. In some aspects, the treatment protocol can include providing a subject a cell expressing one or immunotherapeutic agents, such as a T cell expressing a TCR, a CAR, a cytokine and/or a fusion protein.

### Therapeutic Modality

In some aspects, the RTCs (e.g., RTCs before or after expansion to 3D culture) and/or the subjects described herein can be treated with a therapeutic modality. As used herein, “therapeutic modality” refers to the administration of one or more of thermal, mechanical, electromagnetic or light energies for therapeutic purposes.

In some aspects, the therapeutic modality can be radiotherapy. As used herein, radiotherapy or radiation therapy, is the treatment of cancer and other diseases with ionizing radiation. Radiation therapy used according to the present disclosure can include, but is not limited to, the use of γ-rays, X-rays, and/or the directed delivery of radioisotopes to cancer cells. Other forms of DNA damaging factors are also contemplated such as microwaves and UV-irradiation. Dosage ranges for X-rays range from daily doses of 50 to 200 roentgens for prolonged periods of time (3 to 4 weeks), to single doses of 2000 to 6000 roentgens. Dosage ranges for radioisotopes can depend on the half-life of the isotope, the strength and type of radiation emitted, and the uptake by the cancer cells. Radiotherapy can include the use of radiolabeled antibodies to deliver doses of radiation directly to the cancer cells (radioimmunotherapy). This approach can be used to minimize the risk of radiation damage to healthy cells. Radiotherapy can also include use of radiosensitizers to enhance damage to tumor cells and radioprotectors to protect normal cells from the effects of radiation.

In some aspects, the therapeutic modality can be hyperthermia. Hyperthermia is a type of treatment in which cells and/or body tissue can be heated to a temperature as high as 113 degrees Fahrenheit to help damage or kill cancer cells including RTCs. Hyperthermia can be achieved using probes that make microwaves, radio waves, ultrasound, and/or lasers.

In some aspects, the therapeutic modality can be photodynamic therapy and/or a surgery.

### Equivalents and Scope

The compositions and methods are more particularly described below and the Examples set forth herein are intended as illustrative only, as numerous modifications and variations therein will be apparent to those skilled in the art. The terms used in the specification generally have their ordinary meanings in the art, within the context of the compositions and methods described herein, and in the specific context where each term is used. Some terms have been more specifically defined herein to provide additional guidance to the practitioner regarding the description of the compositions and methods.

As used herein, the term “and/or” includes any and all combinations of one or more of the associated listed items. As used in the description herein and throughout the claims that follow, the meaning of “a”, “an”, and “the” includes plural reference as well as the singular reference unless the context clearly dictates otherwise. The term “about” in association with a numerical value means that the value varies up or down by 5%. For example, for a value of about 100, means 95 to 105 (or any value between 95 and 105).

All patents, patent applications, and other scientific or technical writings referred to anywhere herein are incorporated by reference herein in their entirety. The embodiments illustratively described herein suitably can be practiced in the absence of any element or elements, limitation or limitations that are specifically or not specifically disclosed herein. Thus, for example, in each instance herein any of the terms “comprising,” “consisting essentially of,” and “consisting of” can be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the claims. Thus, it should be understood that although the present methods and compositions have been specifically disclosed by embodiments and optional features, modifications and variations of the concepts herein disclosed can be resorted to by those skilled in the art, and that such modifications and variations are considered to be within the scope of the compositions and methods as defined by the description and the appended claims.

Any single term, single element, single phrase, group of terms, group of phrases, or group of elements described herein can each be specifically excluded from the claims.

Whenever a range is given in the specification, for example, a temperature range, a time range, a composition, or concentration range, all intermediate ranges and subranges, as well as all individual values included in the ranges given are intended to be included in the disclosure. It will be understood that any subranges or individual values in a range or subrange that are included in the description herein can be excluded from the aspects herein. It will be understood that any elements or steps that are included in the description herein can be excluded from the claimed compositions or methods

In addition, where features or aspects of the compositions and methods are described in terms of Markush groups or other grouping of alternatives, those skilled in the art will recognize that the compositions and methods are also thereby described in terms of any individual member or subgroup of members of the Markush group or other group.

The following are provided for exemplification purposes only and are not intended to limit the scope of the embodiments described in broad terms above.

### EXAMPLES

### Example 1: Transcriptomic Profiling of RTCs

For single cell transcriptomic profiling, RTCs are dropped into wells pre-filled with lysis buffer and RNAse inhibitor. The plates are then frozen until further processing. Primers are added to individual wells to mark which cells are in each well. This allows matching the morphological and holographic profiling data with the transcriptomic data. After the primers are annealed, reverse transcriptase is performed before pooling the samples for cDNA amplification. The sample are processed using magnetic beads and quality control is performed on a bioanalyzer. The sample library is prepared from these samples sent for RNA sequencing.

### Example 2: Morphological and Holographic Profiling of RTCs

Holographic cell images are acquired from a sample flown through a microfluidic device. Machine learning is used to identify cells with similar characteristics to cancer cells among the healthy cells. Morphological/optical information of individual cells and populations of cells such as size, optical density, nuclear to cytoplasm ratio, texture complexity, and neural network-derived features are computed from the images. The association between the morphological and holographic profiling and the corresponding molecular profiling together with treatment outcomes for each patient will be tracked. The data are collected from multiple RTCs samples (e.g., RTCs from a multitude of patients having a specific type of cancer, RTCs from cancer patients all having a specific type of treatment, RTCs from cancer patients in general, RTCs from patients all having a related type of cancer (i.e., all types or kidney cancers or all types of breast cancers), etc.) to prepare a reference RTC profile library.

### Example 3

FIG. 1 provides an example of the preparation of an RTC profile library and the preparation of patient RTC's that are to be compared to the RTC profile library. First, RTC's can be obtained from a patient using, for example, dialysis. RTCs can be enriched using, for example, inertial enrichment. Cells can be sorted with the holographic imaging to select RTC's. At this step RTCs can be profiled (e.g., scRNA-seq, genomic profiling, transcriptomic profiling and/or proteomic profiling) or otherwise profiled (e.g., morphological profiled) to create an RTC expression profile. At the same time, RTCs can be cultured for organoid, spheroid, microtumor, or ex vivo tumor growth. The morphological profile of the organoids, spheroids, microtumors, or ex vivo tumors can be obtained.

The organoids, spheroids, microtumors, or ex vivo tumors can be passaged and then: (i) bio-banked for later use; (ii) genomic profiled, transcriptomic profiled, proteomic profiled, and/or otherwise suitably profiled; and/or (iii) tested for drug or other therapy response. Organoids, spheroids, microtumors, or ex vivo tumors can be determined to be sensitive or resistive to the drug/therapy and then each can be genomic profiled, transcriptomic profiled, proteomic profiled, and/or otherwise suitably profiled. The results can be loaded to a reference RTC profile library.

Where the organoids, spheroids, microtumors, or ex vivo tumors are derived from a patient, the genomic profile, transcriptomic profile and/or proteomic profile can be compared to the RTC profile library. Where the genomic profile, transcriptomic profile and/or proteomic profile of the patient's cells are similar to an RTC profile library for sensitive cells (that is, cells that are sensitive to a particular drug or therapy), then the patient can be referred to that particular drug or therapy. Where the genomic profile, transcriptomic profile and/or proteomic profile of the patient's cells are similar to an RTC profile library for resistive cells (that is, cells that are resistant to a particular drug or therapy), then the patient can be advised to avoid that particular drug or therapy.

We claim:

1. A method of preparing a reference released tumor cell (RTC) profile library comprising:
(i) isolating or enriching one or more released tumor cells (RTCs) from one or more biological samples one or more subjects to form isolated or enriched RTCs;
(ii) expanding each of the one or more isolated or enriched RTCs in three-dimensional (3D) cell culture to form expanded RTCs;
(iii) contacting or treating the expanded RTCs with one or more therapeutic agents or therapeutic modalities to identify expanded RTCs that are responsive to the one or more therapeutic agents or therapeutic modalities and expanded RTCs that are non-responsive to the one or more therapeutic agents or therapeutic modalities; and
(iv) conducting molecular profiling, morphologic profiling, or both molecular profiling and morphologic profiling of the expanded RTCs that are responsive and the expanded RTCs that are non-responsive to the at least one therapeutic agent or therapeutic modality, thereby generating a reference RTC profile library.

2. The method of claim 1, wherein the enriched RTCs are enriched by one or more of a physical enrichment method and a biological enrichment method prior to step (ii).

3. The method of claim 1, wherein step (iv) further comprises conducting molecular profiling of the isolated RTCs and the RTCs expanded in 3D cell culture.

4. The method of claim 1, wherein the at least one therapeutic agent is a small molecule, a biological agent, or an immunotherapeutic agent.

5. The method of claim 1, further comprising incorporating at least one immune cell type isolated from the biological sample, into the 3D cell culture.

6. The method of claim 1, wherein the expanded RTCs are expanded in 3D cell culture as organoids, spheroids, microtumors, or ex vivo tumors.

7. The method of claim 1, wherein the isolated or enriched RTCs are circulating tumor cells (CTCs) or disseminated tumor cells (DTCs).

8. The method of claim 1, wherein the morphological profiling comprises holographic profiling..

9. The method of claim 1, wherein the molecular profiling is genomic profiling, transcriptomic profiling, or proteomic profiling.

10. The method of claim 9, wherein the transcriptomic profiling is single cell RNA sequencing.

11. The method of claim 9, wherein the transcriptomic profiling is bulk RNA sequencing.

12. The method of claim 9, wherein the genomic profiling is single cell DNA sequencing.

13. The method of claim 9, wherein the genomic profiling is bulk DNA sequencing.

14. The method of claim 9, wherein the proteomic profiling is single cell proteomics profiling.

15. The method of claim 9, wherein the proteomic profiling is bulk proteomics profiling.

16. The method of claim 1, wherein the biological sample is a blood sample, a serum sample, a plasma sample, a saliva sample, a fecal sample, a urine sample, a cerebrospinal fluid sample, an interstitial fluid sample, a peritoneal fluid sample, a pleural fluid sample, a cyst fluid sample, a semen sample, a mucous sample, a sputum sample, an intestinal fluid sample, or a lymphatic fluid sample.

17. The method of claim 1, wherein the biological sample a fine-needle aspirated tissue sample or a bone marrow sample.

18. A method of determining a treatment protocol for a cancer patient:
(i) isolating RTCs from a biological sample of the cancer patient to form isolated RTCs;
(ii) conducting molecular profiling, morphologic profiling, or both molecular profiling and morphologic profiling of the isolated RTCs from the cancer patient to establish a molecular profile, a morphologic profile, or both molecular profile and morphologic profile of the isolated RTCs; and
(iii) comparing the molecular profile, morphologic profile, or both molecular profile and morphologic profile of the isolated RTCs from the cancer patient to the reference RTC profile library of claim 1 to identify a treatment protocol.

19. The method of claim 18, wherein the isolated RTCs from the biological sample of the cancer patient are enriched by one or more of a physical enrichment method or a biological enrichment method prior to step (ii).

20. The method of claim 18, wherein the RTCs from the cancer patient are expanded as RTCs in 3D cell culture to form isolated, expanded RTCs prior to step (ii).

21. The method of claim 20, wherein the isolated, expanded RTCs are expanded in 3D cell culture as organoids, spheroids, microtumors, or ex vivo tumors prior to step (ii).

22. The method of claim 18, wherein the isolated RTC is a circulating tumor cell (CTC) or a disseminated tumor cell (DTC).

23. The method of claim 18, further comprising incorporating at least one immune cell type isolated from the biological sample, into the 3D cell culture.

24. The method of claim 18, wherein the molecular profiling is genomic profiling, transcriptomic profiling, or proteomic profiling.

25. The method of claim 24, wherein the transcriptomic profiling is single cell RNA sequencing.

26. The method of claim 24, wherein the transcriptomic profiling is bulk RNA sequencing.

27. The method of claim 24, wherein the genomic profiling is single cell DNA sequencing.

28. The method of claim 24, wherein the genomic profiling is bulk DNA sequencing.

29. The method of claim 24, wherein the proteomic profiling is single cell proteomics profiling.

30. The method of claim 24, wherein the proteomic profiling is bulk proteomics profiling.

31. The method of claim 18, wherein the biological sample is a blood sample, a serum sample, a plasma sample, a saliva sample, a fecal sample, a urine sample, a cerebrospinal fluid sample, an interstitial fluid sample, a peritoneal fluid sample, a pleural fluid sample, a cyst fluid sample, a semen sample, a mucous sample, a sputum sample, an intestinal fluid sample, or a lymphatic fluid sample.

32. The method of claim 18, wherein the biological sample is a fine-needle aspirated tissue sample or a bone marrow sample.

33. The method of claim 18, further comprising comparing the molecular profile of the isolated RTCs from the cancer patient to a molecular profile of a reference cancer cell or a population thereof.

34. The method of claim 33, wherein the reference cancer cell is a cancer cell line cell, a cancer biopsy cell, or a patient-derived xenograft cell.

35. The method of claim 18, wherein the morphological profiling comprises holographic profiling.
